(19) United States
(12) Patent Application Publication
(10) Pub. No.: US 2005/0008580 A1
(43) Pub. Date: Jan. 13, 2005

(54) Hemophilia treatment by inhalation of coagulation factors

(75) Inventors: David K. Gong, Cupertino, CA (US); Jayne E. Hastedt, San Carlos, CA (US); Robert G. Schaub, Pelham, NH (US); Nicholas W. Warne, Andover, MA (US); Andrew J. Dorner, Cambridge, MA (US); Chandra A. Webb, Pelham, NH (US); James C. Keith, Andover, MA (US)

Correspondence Address: JENKENS & GILCHRIST, 1401 MCKINNEY, SUITE 2600, HOUSTON, TX, 77010, US

(73) Assignees: Wyeth, Madison, NJ (US); Nektar Therapeutics, San Carlos, CA (US)

(21) Appl. No.: 10/820,656

(22) Filed: Apr. 8, 2004

Related U.S. Application Data
(60) Provisional application No. 60/461,460, filed on Apr. 9, 2003.

(51) Int. Cl. A61L 9/04; A61K 38/37

(52) U.S. Cl. 424/46; 514/12

(57) ABSTRACT

Hemophilia treatment by the inhalation of coagulation factors. Dry powder Factor IX is aerosolized to a mass median aerodynamic diameter of 4 μm or less, with at least 90% monomer content, at least 80% activity level, and 10% water or less. The aerosol is slowly, and deeply inhaled into the lung, and followed by a maximal exhale.

### PRIOR RELATED APPLICATIONS

This application claims priority to provisional application U.S. Ser. No. 60/461,460 filed Apr. 9, 2003.

### FEDERALLY SPONSORED RESEARCH STATEMENT

Not applicable.

### REFERENCE TO MICROFICHE APPENDIX

Not applicable.

### FIELD OF THE INVENTION

The invention relates to the treatment of hemophilia by inhalation of coagulation factors.

### BACKGROUND OF THE INVENTION

About 450,000 patients worldwide live with bleeding disorders, known as “hemophilias.” Hemophilias are caused by a deficiency of one or more clotting factors in the blood, the lack of which causes prolonged bleeding. Even a minor bruise could trigger internal hemorrhaging. In severe cases, internal bleeding can start without apparent cause, spreading into joints and tissues. Swelling and intense pain usually result and the person with hemophilia suffers throughout their lifespan. There are three main types of hemophilia, each resulting from a mutation in a different protein in the coagulation cascade.

Hemophilia A, sometimes called classical hemophilia, is the most common type of hemophilia, occurring in about 80 percent of patients with congenital factor deficiencies. It is caused by a DNA defect that is carried on the X chromosome and produces deficiencies in Factor VIII. Only one normal X chromosome is necessary to produce adequate levels of Factor VIII. Therefore, nearly all affected patients are men. In most cases, the defective gene is passed down through several generations, but in about 20 percent of cases, the defect arises by spontaneous mutation.

Hemophilia B, also known as Christmas disease, accounts for 12 percent to 15 percent of hemophilia cases and is caused by a deficiency in coagulation Factor IX. Like hemophilia A, hemophilia B is linked to an inherited defect on the X chromosome, and it usually affects the male children of carrier mothers.

Factor XI deficiency accounts for only 2 percent to 5 percent of patients with congenital factor-deficiency states. It is caused by a deficiency in coagulation factor XI, and unlike hemophilia A and B, it is inherited on a chromosome other than the X chromosome and can be passed to both male and female children. Von Willenbrand Disease is yet another form of hemophilia that is prevalent in males and females. There are also some rare forms with other factors that are missing such as Factor V, X and XIII.

Current therapy for these hemophilic patients consists of intravenous (IV) administration of coagulation factors given prophylactically to prevent bleeding or “on demand” for hemorrhagic events. Treatment can be administered in a clinic or at home, however, inability to establish venous access can make therapy very difficult at either location. Extravascular administration of coagulation factors could circumvent this difficulty. The subcutaneous (SC), intramuscular (IM), and intraperitoneal (IP) routes of administration achieve therapeutic levels, but needles are still typically used for delivery (1).

Inhalation therapy would provide a “needle-free” route of administration for coagulation factors if therapeutic levels could reach the systemic circulation from the airways. The respiratory system is an attractive route for systemic delivery of proteins or peptides that cannot withstand proteolysis in the gastrointestinal tract or as an alternative to IV, SC, IM, or IP routes. For treatment of hemophilia, the respiratory tract offers several advantages. First, a coagulation factor administered by inhalation only needs to transit a relatively short distance between the pulmonary epithelium and the systemic circulation. Second, the smaller airways and alveoli have a large surface area composed of a highly permeable and absorptive membrane. Third, the alveoli harbor a huge vascular bed through which several liters of blood flow per minute. Fourth, the lung has relatively low enzymatic activity and airway mucous and the thin surfactant aqueous layer of the alveoli contain high concentrations of protease inhibitors (2). This environment might make degradation of a protein less likely and could afford proteins such as F.IX, F.VIII and F.XI at least some protection from degradation during transit to the systemic circulation (2, 3).

The most important parameter that defines the site of deposition of aerosol proteins within the respiratory tract is the particle characteristics of the aerosol. Behavior of aerosol droplets is dependent on their “mass median aerodynamic diameter” (MMAD), which is a function of particle size, shape, density and charge. Air velocities within the airways is also an important attribute.

Strict control of MMAD of the particles ensures reproducibility of aerosol deposition and retention within desired regions of the respiratory tract. Good distribution throughout the lung requires particles with an aerodynamic diameter between 1 and 5 μm. Very small particles (<1 μm) are exhaled during normal tidal breathing. Particles that are 3 μm are targeted to the alveolar region, and particles that are greater than 6 μm are deposited in the oropharynx.

Optimal management of most diseases requires accurate dosing of the therapeutic compound. Pulmonary drug administration imposes stringent requirements on the delivery device; this is because the particle size of the powder or droplet greatly influences the delivery site, and thus the degree of drug absorption from the lungs.

The devices that are currently available for pulmonary drug administration were mostly developed to achieve local effects of the drug in the conducting airways, such as in asthma. These devices include nebulizers, metered-dose inhalers (MDIs) and dry-powder inhalers (DPIs).

Use of nebulizers to administer biopharmaceutical agents has many important limitations. Such drugs are often very unstable in aqueous solutions, and are easily hydrolyzed. In addition, the process of nebulization exerts high shear stress on the compounds, which can lead to protein denaturation. This is a particular problem because 99% of the droplets generated are recycled back into the reservoir to be nebulized during the next dosing (6). Furthermore, the droplets produced by nebulizers are heterogeneous, which results in poor drug delivery to the lower respiratory tract. The propellants (chlorofluorocarbons and, increasingly, hydrofluoroalkanes) used to atomize the protein solution in MDI's can also contribute to protein denaturation.

A promising alternative to nebulizers and MDIs is the DPI, which delivers the protein in dry form. Like MDIs, most DPIs that are currently approved are made for pulmonary drug administration of locally acting drugs for the management of asthma and chronic obstructive pulmonary diseases, such as anti-asthmatic agents.

Most efforts at systemic therapy by inhalation routes of administration have been directed to diabetes. Until recently, researchers believed that insulin delivered noninvasively was associated with too low a bioavailability to offer a realistic clinical approach. However, a growing body of evidence suggests that inhaled insulin is an effective, well-tolerated, noninvasive alternative to injected insulin, and inhalation therapy for insulin is in phase 3 clinical trials.

Insulin is made of an alpha and beta subunit that originates from a single gene. The functional recombinant enzyme is about 5.9-6.9 KD, although there is evidence to suggest that under physiological conditions native insulin exists as a hexamer of about 31.2-32.8 KD. Insulin is, therefore, a very small protein, which may account for its success in inhalation delivery. Other metabolic hormones that have been delivered by inhalation therapy are also small: Calcitonin (35 KD), HGH (22 KD), TSH alpha (13 KD), TSH beta (15-16 KD), FSH (36 KD) and somatostatin (2 KD). Heparin (20 KD) has also been tested by inhalation delivery as an anti-coagulation agent. In addition to size, the degree of bioavailability may also depend on a therapeutic protein's susceptibility to hydrolytic enzymes in the lung. Little effort has been directed to inhalation therapy of larger proteins, probably due to the difficulty in successfully aerosolizing, delivering and absorbing larger proteins.

To our knowledge, no one has succeeded in the pulmonary delivery of coagulation proteins, presumably due to their large size and their notorious instability in solution. Glycosylated Factor IX is 55 KD, Factor VIII is 200 KD, and Factor XI is 140-150 kD, thus these proteins are considerably larger than those discussed above. Gupta (29) attempted the pulmonary delivery of coagulation factors, but found that human Factor IX was denatured during nebulization and hypothesized that this was due to shear forces imposed by the nebulizers or the large air water interface produced during the process.

Until the work described herein, no one has successfully aerosolized and delivered proteins as large and as delicate as Factor IX to the pulmonary system. Further, until now no one has successfully treated hemophilia by inhalation therapy.

### SUMMARY OF THE INVENTION

The invention generally relates to a method of treating hemophilia, with an aerosolized Factor IX (F.IX), wherein the aerosolized F.IX has a mass median aerodynamic diameter (MMAD) of between 2 and 4 μm, a fine particle fraction percent less than 3.3 μm (FPF %<3.3 μm) of at least 50%, is at least 80% monomeric protein, an after-aerosolization activity/pre-aerosolization activity of at least 80%; and is a dry powder having less than 20% water (wt/wt). The aerosol is slowly maximally inhaled to deposit the F.IX in the deep lung tissue, followed by maximal exhalation.

Because the inhaled F.IX appears to be sequestered in the lung for some period of time after inhalation administration, the method is also applicable to the prophylactic or preventative treatment of hemophilic bleeding in advance of a bleeding event. Thus, weekly or biweekly application of F.IX produces a depot effect, allowing sufficient F.IX to remain accessible to prevent bleeding even 2-4 days after administration. Thus a weekly or biweekly application is prophylactic.

In preferred embodiments, the MMAD is 2 to 5 μm, 2.8 to 3.6 μm, or 3-3.5 μm, the FPF %<3.3 μm is at least 60% or 64% and the monomer content is at least 95% or 97%. The after-aerosolization activity/pre-aerosolization activity is at least 85%, preferably 90 or 95%. Water content is preferably very low, as low as 10 or 5%. Further preferred is a method whereby the F.IX is aerosolized without alcohol, as alcohol appears to negatively affect long term storage of the spray dried powders. Also preferred is the use of recombinant F.IX.

A preferred embodiment uses a surface active di- or tripeptide as an excipient. Di-leucyl containing tripeptides for use in the invention are tripeptides having the formula, X-Y-Z, where at least X and Y or X and Z are leucyl residues. Especially preferred is a di- or tri-leucine excipient, where the di- or tri-leucine/F.IX ratio is about 0.5-1.5 wt/wt or 45/40 wt/wt.

Compositions of aerosolized F.IX, and blister packs containing fine, dry F.IX are also included within the scope of the invention.

“Leucine”, whether present as a single amino acid or as an amino acid component of a peptide, refers to the amino acid leucine, which may be a racemic mixture or in either its D- or L-form, as well as modified forms of leucine (i.e., where one or more atoms of leucine have been substituted with another atom or functional group) in which the dispersibility-enhancing effect of the modified amino acid or peptide is substantially unchanged over that of the unmodified material.

“Dipeptide”, refers to a peptide composed of two amino acids. “Tripeptide” refers to a peptide composed of three amino acids.

A “surface active” material is one having surface activity (measured, e.g., by surface tensiometry), as characterized by its ability to reduce the surface tension of the liquid in which it is dissolved. Surface tension, which is associated with the interface between a liquid and another phase, is that property of a liquid by virtue of which the surface molecules exhibit an inward attraction.

“Dry powder” refers to a powder composition that typically contains less than about 20% moisture, preferably less than 10% moisture, more preferably contains less than about 5-6% moisture, and most preferably contains less than about 3% moisture, depending upon the particular formulation.

A dry powder that is “suitable for pulmonary delivery” refers to a composition comprising solid capable of being (i) readily dispersed in/by an inhalation device and (ii) inhaled by a subject so that a portion of the particles reach the lungs. Such a powder is considered to be “respirable.” “Aerosolized” particles are particles which, when dispensed into a gas stream remain suspended in the gas for an amount of time sufficient for at least a portion of the particles to be inhaled by the patient, so that a portion of the particles reach the lungs.

### BRIEF DESCRIPTION OF THE DRAWINGS

FIG. 1. F.IX activity in hemophilia B dogs following a single-dose of rF.IX given intravenously or intratracheally. rF.IX given IV (200 IU/kg) produced an immediate and biphasic response in F.IX activity. rF.IX given IT (200 or 1000 μIU/kg) produced detectable F.IX activity levels that were delayed in onset, beginning at 8 h. F.IX activity was detected for at least 72 h with the IV dose and both IT doses. Administration of the 200 and 1000 IU/kg IT doses achieved comparable therapeutic levels that were less than that achieved with the 200 IU/kg IV dose. Each data point represents the mean±standard deviation calculated from 3 dogs, except for the 18 h time point in the IV group, which represents data from 2 dogs.

FIG. 2. F.IX antigen in hemophilia B dogs following a single-dose of rF.IX given intravenously or intratracheally. The F.IX antigen essentially mirrors the activity assays shown in FIG. 1 except that the duration of detection appears shorter. This apparent shorter duration is probably due to the sensitivity of this assay.

FIG. 3. Cumulative total amount of rF.IX absorbed after intratracheal administration of 200 IU/kg or 1000 IU/kg to hemophilia B dogs. The cumulative amount absorbed over time for both the 200 IU/kg and the 1000 IU/kg IT dose groups appears similar. The total amount of rF.IX absorbed is approximately 21 IU/kg and 37 IU/kg for the 200 IU/kg and 1000 IU/kg IT groups respectively. These data are consistent with a non-proportional increase in the amount absorbed between the two dose groups (see FIG. 4).

FIG. 4. Cumulative amount of rF.IX absorbed as a percent of total dose administered in hemophilia B dogs that received 200 IU/kg or 1000 IU/kg intratracheally. The percent of total dose absorbed calculated by deconvolution analysis was approximately 10.2% and 3.7% for the 200 IU/kg and 1000 IU/kg dose groups, respectively.

FIG. 5. APTT Shortening Following rF.IX Inhalation in a Naïve Hemophilia B Dog.

FIG. 6. WBCT Shortening Following rF.IX Inhalation in a Naïve Hemophilia B Dog.

FIG. 7. Mean Corrected rF.IX Antigen Concentration Time Curve for Tolerized Hemophilia B Dogs (n=3) Receiving rF.IX (50 IU/kg) by Inhalation.

FIG. 8. Cumulative Amount of rF.IX Absorbed After Inhalation in Tolerized Hemophilia Dogs (n=4) as Determined by Antigen Assay. Dogs are C22 (top line 4), C20 (line 3), C25 (line 2), C26 (bottom line 1).

### DESCRIPTION OF EMBODIMENTS OF THE INVENTION

The present invention is exemplified with respect to human recombinant Factor IX. However, with the knowledge gained herein, the aerosolization of larger coagulation factors, such as F.VIII and F.XI will be attempted. These factors are even larger than F.IX, and may be more difficult to administer by inhalation therapy. However, it may be possible to administer a truncated, functional fragment thereof.

The invention provides a method of treating hemophilia by inhalation therapy of dry, aerosolized coagulation factor powders having an MMAD of less than 3.5 um, an FPF of greater than 0.50 and greater than 95% monomer content. Such powders allow for localization in pulmonary tissue resulting in a slow release of active coagulation factor ideal for treatment of hemophilia.

### EXAMPLE 1

### Treatment of Hemophila via Intratracheal Administration of Liquid Factor IX

For proof of concept, we deposited liquid human recombinant Factor IX (rF.IX) intratracheally (IT) in a hemophilia B dog model. If the liquid IT rF.IX demonstrated bioavailability, then we would proceed further and test an aersolized dry powder form of the protein in the same model system.

Hemophilia B dogs: Hemophilia B dogs from the closed colony at the Francis Owen Blood Research Laboratory at the University of North Carolina in Chapel Hill were used in this study. The causative molecular defect in these dogs is a missense point mutation (G to A at nucleotide 1477) in the catalytic domain of the Factor IX molecule, resulting in a complete absence of circulating F.IX (6). This strain of hemophilia B dogs has neither detectable F.IX activity in functional assays nor antigen by ELISA or immunoblot (7, 8). All animals were treated according to standards in the Guide for the Care and Use of Laboratory Animals (National Institutes of Health publication No. 85-23). The Institutional Animal Care and Use Committee approved all experiments.

Human recombinant Factor IX (rF.IX): rF.IX was prepared by Genetics Institute, Inc., Andover, Mass. (now Wyeth), as previously described (9-11). This preparation was highly concentrated with a F.IX activity of approximately 12,500 IU/ml and a protein concentration of approximately 39 mg/ml. rF.IX was stored at −80° C. in its vehicle formulation buffer until administered (12).

In vivo experiments: Nine hemophilia B dogs were randomly assigned to one of three treatment groups: 200 IU/kg (n=3) or 1000 IU/kg (n=3) intratracheal administration or 200 IU/kg intravenous infusion (n=3). Dogs receiving intratracheal doses of rF.IX were sedated with propofol or medetomidine hydrochloride and maintained under a surgical plane of anesthesia with isofluorane (2-4% via nose cone) during the procedure, if indicated. For intratracheal (IT) dosing, an endoscope was inserted into the left or right bronchial tree. A 7 French (˜2 mm in diameter) triple lumen pulmonary artery catheter was inserted under endoscopic guidance into the appropriate bronchus. The dose (in a 1 ml volume) was evenly divided between the right and left bronchi and infused over approximately two minutes. After rF.IX infusion, the catheter was flushed with 2 ml of 0.9% saline. In comparative experiments, the intravenous (IV) dose was injected as a bolus into the cephalic vein over a period of 2-3 min.

Sampling protocol: Blood samples were taken prior to and after administration of rF.IX at the following time points: 0, 5, 15, 30 min, and 1, 2, 4, 8, 12, 18, 24, 36, 48, and 72 h. Whole blood was drawn by venipuncture and collected in 4% sodium citrate, at a final concentration of 1 part anticoagulant to 8 parts whole blood. Plasma was prepared and frozen at −80° C. until analyzed. Serum samples for anti-F.IX antibody titers were taken prior to rF.IX administration and afterwards on days 5, 10, 15, and 28. Whole blood for performing the whole blood clotting time was obtained from selected dogs in each group, 2 h post treatment. Complete blood counts (CBC) were performed on dogs receiving rF.IX pretreatment and 48-72 h post treatment. Thoracic radiographs were obtained at the same timepoints on dogs from the IT group. At the end of the study, the dogs were killed by an overdose of pentobarbital and necropsies were performed.

Whole blood clotting time (WBCT): The WBCT was performed as previously described (7, 13-15). The WBCT is typically greater than 50 min in untreated hemophilia B dogs from the Chapel Hill colony (14, 15). The reference range for WBCT in normal, healthy dogs in this colony is 8 to 12 min. The WBCT was determined at 2 h following treatment in three dogs selected from the IT groups. It shortened to 23.5 min in one dog that received 200 IU/kg IT and 21.5 min in one of the two dogs tested that received 1000 IU/kg IT. The WBCT in all three dogs from the IV group corrected to 9.5 min when assayed at 2 h post treatment.

F.IX activity: F.IX clotting activity was determined using a modified Activated Partial Thromboblastin Time (APTT) test on a Multi-Discrete Analyzer 180 (MDA-180, ORGANON TEKNIKA™, Durham, N.C.) (4). Control standards consisted of dilutions prepared from 1 ml of pooled F.IX-deficient canine plasma containing 1 IU of rF.IX.

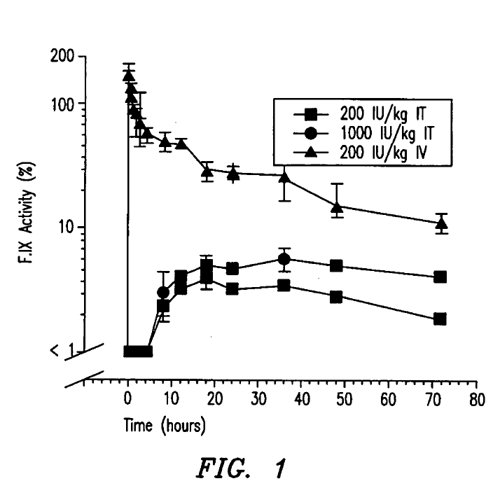

F.IX activity (FIG. 1) was not detected in any of the dogs prior to infusion of rF.IX. Following IT administration F.IX activity was detected at 8 h post infusion and was still measurable at 72 h. Little difference in the plasma level was noted between the two IT doses. Intravenous administration of rF.IX produced an immediate and biphasic response as reported in previous studies (4). F.IX activity was detected at 5 min post infusion and through 72 h and maximum activity was reached by IV administration.

F.IX antigen concentration: The F.IX antigen concentration was determined using a double monoclonal antibody sandwich enzyme linked immunosorbant assay (ELISA) (12). The lower limit of the ELISA in this study was ˜38 ng/ml. All values below this limit were assumed to be less than 1 ng/ml.

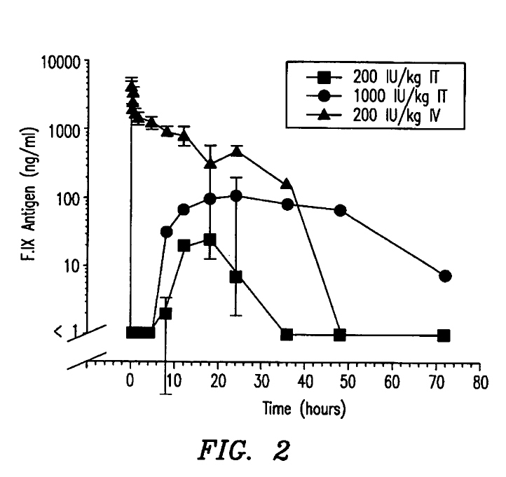

F.IX antigen concentration (FIG. 2) followed a similar pattern as seen with F.IX activity in all three groups. F.IX antigen was detected in the first blood samples (5 min) in the IV group, but not until 8 h in both IT groups. As expected, the highest detectable antigen concentrations were found in the IV group.

Pharmacokinetic analysis: The pharmacokinetic analyses were performed on the activity time data for both the IV and IT groups. A two compartment model (WinNonlin, PHARSIGHT CORP.™, Mountain View Calif.) best described the IV data (model 8), and a one compartment model with a lag time best described the IT data (model 4). Numerical deconvolution analysis was also performed on the data to understand the rate and extent of absorption (16).

Table 1 comparing the two IT groups to the IV group showed that the highest mean maximum plasma concentration (Cmax) occurred with IV administration (157.3±29.3 IU/dl). The mean values for Cmax in the 200 IU/kg and 1000 IU/kg IT groups were 4.7±0.5 IU/dl and 6.5±0.5 IU/dl, respectively. The total exposure after IV administration (Area under the curve; AUC_{0-∞}) was 2716+/−164 IU/dL×hr. In comparison the total exposure after IT administration was 306+/−20.8 IU/dL×hr and 666+/−127 IU/dL×hr for the 200 IU/kg and 1000 IU/kg IT groups respectively. The mean T½ was 24.2±10.7, 30.7±5.3, and 46.4±29.2 h for the IV, 200 IU/kg IT and 1000 IU/kg IT groups, respectively.

TABLE 1
Pharmacokinetic Analysis Following IT or IV Administration of rF.IX

| Group | Cmax | Tmax | T1/2 | AUC | Bioavailability % |
|---|---|---|---|---|---|
| 200 IU/kg IT | 4.7 ± 0.5 | 21.1 ± 3.4 | 30.7 ± 5.3 | 306 ± 20.8 | 11.3 ± 0.8 |
| 1000 IU/kg IT | 6.5 ± 0.5 | 30.0 ± 6.3 | 46.4 ± 29.2 | 666 ± 127 | 4.9 ± 1.1 |
| 200 IU/kg IV | 157.3 ± 29.3 | — | 24.2 ± 10.7 | 2,716 ± 164 | — |

It should be noted that the halflife appears longer on the F.IX activity curve (FIG. 1) than on the F.IX antigen curve (FIG. 2). However, these samples were prepared concurrently. The F.IX ELISA was determined to have a threshold sensitivity of 38 ng/ml. Since the activity assays are more sensitive than this ELISA, the activity assays most likely are a more accurate representation of F.IX clearance. The time to maximum concentration (Tmax in hours) was similar between the two IT doses, 21.1±3.4 and 30.0±6.3 respectively. The bioavailability after IT administration was 11.3% for the 200 IU/kg IT group and 4.9% for the 1000 IU/kg IT group.

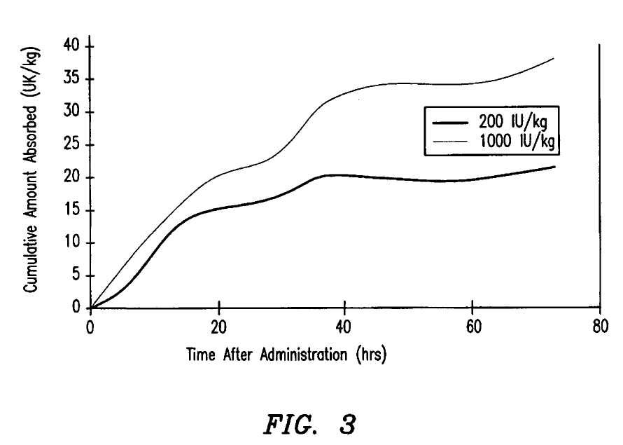

The cumulative amount absorbed over time for both the 200 IU/kg and the 1000 IU/kg IT dose groups shown in FIG. 3 indicates that the absorption rate for the 2 doses was similar since the slopes of the two curves are similar. The total amount absorbed, however, was different for the two doses. For the 200 IU/kg IT dose the total amount absorbed was approximately 21 IU/kg and for the 1000 IU/kg IT group the total amount absorbed was approximately 37 IU/kg. Therefore there was a non-proportional increase in the amount absorbed between the two dose groups.

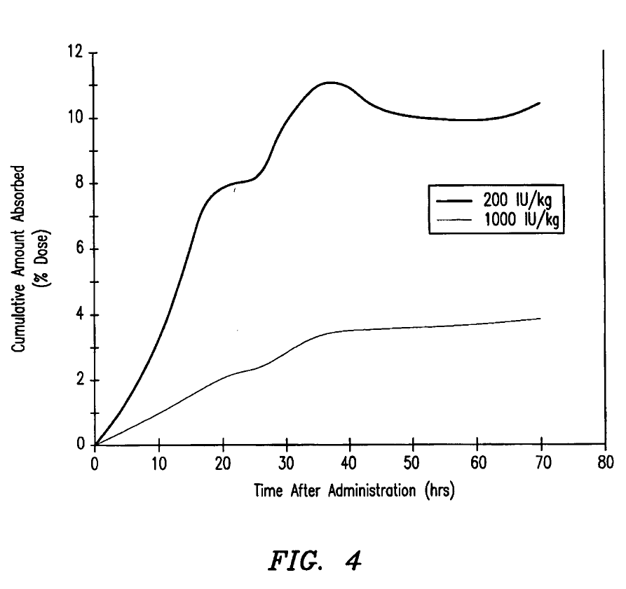

This observation may be further noted in FIG. 4. The percent of total dose absorbed calculated by deconvolution analysis was approximately 10.2% and 3.7% for the 200 IU/kg and 1000 IU/kg IT dose groups, respectively. These data are similar to the bioavailability values for the 2 groups calculated by comparison of the AUC_{0-∞} of 11.3% and 4.9% for the 200 IU/kg and 1000 IU/kg dose groups, respectively.

Anti-human F.IX antibody analysis: Titers for anti-human F.IX antibody in canine serum from treated dogs were determined using ELISA that is specific for canine anti-human F.IX IgG antibodies (12). The antibody titer for a given dog is arbitrarily defined as the plasma sample dilution that produces a two-fold increase in an optical density (OD) signal when compared to a negative control. The threshold of sensitivity for this assay is 25 arbitrary units.

Adult hemophilia B dogs routinely develop an antibody to the human F.IX. Anti-human F.IX antibody titers were detected in all of the dogs from both IT groups by day 10 following administration (Table 2). Two of the 3 dogs from the IV group had detectable antibody titers at this same time point. Anti-human F.IX antibody titers were detected in all of the dogs by day 15 which persisted through day 28 of the study.

TABLE 2
Anti-human F.IX Antibody Titers Following IT and IV Administration of rF.IX

| Day | 200 IU/kg IT | 200 IU/kg IT | 200 IU/kg IT | 1000 IU/kg IT | 1000 IU/kg IT | 1000 IU/kg IT | 200 IU/kg IV | 200 IU/kg IV | 200 IU/kg IV |
|---|---|---|---|---|---|---|---|---|---|
| pre | <25 | <25 | <25 | <25 | <25 | 54 | 74 | <25 | <25 |
| 5 | <25 | <25 | <25 | 335 | <25 | <25 | <25 | <25 | <25 |
| 10 | 79 | 623 | 617 | 6265 | 357 | 5841 | 1124 | <25 | 120 |
| 15 | 86 | 1076 | 408 | 11,109 | 775 | 12,600 | 1546 | 230 | 1502 |
| 28 | <25 | 365 | 327 | 5258 | 141 | 2058 | 1213 | 1429 | — |

Clinical profile and immune response: Intratracheal administration of concentrated rF.IX has not been previously attempted. Therefore the dogs were monitored clinically for any adverse responses. No cough was noted in the 200 IU/kg dose IT dogs or the dogs receiving rF.IX IV. Dogs that received 1000 IU/kg dose IT had a mild, transient cough approximately at 45 min to 1 h post infusion, which lasted no longer than 1 h. No abnormal lungs sounds were noted in any of the animals on auscultation. Pre- and post-treatment thoracic radiographs from both IT groups of dogs detected no changes in the appearance of the airways or lung parenchyma. Pre- and 48 or 72 h post-treatment CBCs were unremarkable in all 3 treatment groups. No gross abnormal findings in the trachea or pulmonary parenchyma were noted at necropsy, performed 1 month post treatment.

### EXAMPLE 2

### Aerosolization of Factor IX

Because the tracheal administration of liquid rF.IX proved safe and efficacious, we next attempted to aerosolize rF.IX. Recombinant human Factor IX is a glycoprotein that is 47 kD when unglycosylated and 55 kD when glycosylated. The current pharmaceutical formulation is a lyophilized powder because liquid F.IX tends to be unstable. Even the powder formulation is susceptible to oxidation and degradation when exposed to ambient levels of humidity. Therefore, we chose to use a dry powder aerosolized formulation, in an attempt to minimize the expected instability.

The target aerosol properties for the rF.IX powders were an initial Emitted Dose (ED) value greater than 50%, a Mass Median Aerodynamic Diameter (MMAD) less than 3.5 um and a Fine Particle Fraction (FPF<3.3 μm) of greater than 0.50. Chemically and physically stable powders were classified as having less than 5% loss of purity with respect to the initial spray dry solution characteristics, no visible change in morphology, ED, MMAD and FPF within the target ranges and no change in particle size distribution after exposure to 40° C./0% relative humidity in blister packages for 4 weeks.

Formulations rF.IX solutions for study 1 and study 2 were from Genetics Institute formulated in 10 mM histidine, 260 mM glycine, 1% sucrose, 0.005% Polysorbate-80 at pH 6.8 at concentrations of 12 and 2.26 mg/mL, respectively. Solutions were diafiltered through AMICON™ (MILLIPORE™) units with 1.25 mM sodium citrate buffer at pH 6. Total volume of buffer used for diafiltration was approximately four to five times the original solution volume. Final primary stock solutions concentrations are 12 mg/mL for study 1 and 11.5 mg/mL for study 2 as measured by UV. Formulations were prepared as described in Table 3, using 0.5% total solids in water.

TABLE 3a
Study 1 Formulations (solids only wt/wt %)

| Lot # | *rF.IX | g-rF.IX | NaCitrate | Tri-Leucine | Leucine | Sucrose | Zinc | EtOH |
|---|---|---|---|---|---|---|---|---|
| 8 | 79.3 | 92.6 | 7.4 | 0 | 0 | 0 | 0 | 0 |
| 9 | 70.7 | 82.6 | 7.4 | 10 | 0 | 0 | 0 | 0 |
| 10 | 45.0 | 52.6 | 7.4 | 0 | 40 | 0 | 0 | 0 |
| 11 | 76.1 | 89.0 | 7.4 | 0 | 0 | 3.7 | 0 | 0 |
| 12 | 76.5 | 89.5 | 7.4 | 0 | 0 | 0 | 3.2 | 0 |
| 13** | — | — | — | — | — | — | — | — |

*weight of rF.IX calculated from the weight of glycosylated rF.IX (g-rF.IX) assuming a ratio of 1.17 glycosylated/unglycosylated rF.IX.
**neat: 10 mM Histidine/260 mM Glycine/1% sucrose/0.005% Tween 80

TABLE 3b
Study 2 Formulations (solids only wt/wt %)

| Lot # | *rF.IX | g-rF.IX | NaCitrate | Tri-Leucine | Leucine | Sucrose | Zinc | EtOH |
|---|---|---|---|---|---|---|---|---|
| 3 | 79.3 | 92.6 | 7.4 | 0 | 0 | 0 | 0 | 0 |
| 4 | 78.7 | 92.0 | 7.4 | 0 | 0 | 0 | 0 | 0.05 |
| 5 | 27.9 | 32.6 | 7.4 | 0 | 60 | 0 | 0 | 0 |
| 6 | 45.0 | 52.6 | 7.4 | 40 | 0 | 0 | 0 | 0 |
| 7 | 79.3 | 92.6 | 7.4 | 0 | 0 | 0 | 0 | 0 |

Surface tension measurements were performed at ambient conditions using a KRUSS K12 PROCESSOR TENSIOMETER.™ Water, which was used as a reference was measured at 72.5 mN/m. Solutions were analyzed prior to powder processing. The pH of the solutions was checked at room temperature just prior to spray drying using an ORION™ model 720A pH meter. A 2 point calibration was performed with pH 7.0 and 10.0 standards. Results are provided in Table 4.

TABLE 4
pH and Surface Tension (mN/m)

| Study 1 | | | Study 2 | | |
|---|---|---|---|---|---|
| Lot # | pH | Surface Tension | Lot # | PH | Surface Tension |
| 8 | 6.1 | 33.37 | 3 | 6.4 | 46.64 |
| 9 | 6.1 | 32.76 | 4 | 6.4 | 44.33 |
| 10 | 6.1 | 35.03 | 5 | 6.4 | 49.30 |
| 11 | 6.1 | 33.40 | 6 | 6.4 | 47.64 |
| 12 | 5.6 | 32.44 | 7 | 6.4 | 45.79 |
| 13 | 6.8 | 37.28 | | | |

Aerosolization The 11 formulas were spray dried with a Büchi 190 Mini Spray Dryer (BRINKMAN™) with modified cyclone, atomizer nozzle and powder collection vessel. The atomizer of the Büchi spray dryer was operated with compressed dry air set at 60 psi for study 1 and 40 psi for study 2. The liquid flow rate into the Büchi was 5 mL/min for both studies. The outlet temperature was set at 70° C. for study 1 and 60° C. for study 2. The total air flow through the Büchi was 17.8 scfm. Batch size was 675 to 1,350 mg with yields of 20 to 67% for the 11 lots. The collectors used were 1/2 inch or 1 inch made of borosilicate glass.

Blister Packs The powders were all hand filled by qualified personnel. The powders were transferred into a glovebox with relative humidity less than 5%. The blister configuration used was a P3.05 PVC blister. The powder, 7.5±0.15 mg, was filled into each blister, a lidstock was placed on top and the blister pack was sealed. The sealing temperature was 171° C. (±5° C.) with a dwell time of 1 sec. The blister pack was then die cut to fit into the device.

Stability Tests Aerosol, thermal, physical and chemical tests were performed at initial conditions and after two to three weeks of storage at controlled temperature and relative humidity. Formulation powders were filled into PVC blister packs and assayed for emitted dose, particle size distributions and thermal analyses. Chemical characterizations and scanning electron microscopy (SEM) were performed on bulk aerosol drug powders at initial conditions. All powders were handled in humidity controlled glove boxes with a relative humidity of less than 5%.

Accelerated Storage Conditions Bulk aerosol drug powders for study 1 formulations were desiccated and stored at 2-8° C. and 40° C. (0% RH) and at 25° C. (0, 33 and 75% RH). Bulk aerosol drug powders for study 2 formulations were stored at two temperatures (25° C. and 40° C.) and two relative humidity conditions for both temperature conditions (0 and 75%).

Bulk aerosol drug powder was weighed into borosilicate glass vials in the glove box. For 0% RH stability samples, vials were capped, placed into a foil overwrap pouch with desiccant and heat sealed before storing in temperature controlled chambers. For humidity controlled stability samples, vials were left open and stored in humidity controlled chambers at the appropriate temperature. Samples were analyzed by UV, SDS-PAGE, SE-HPLC and SEM after two or three weeks.

Aerosol Tests A device as described in U.S. Pat. No. 6,257,233 was used to perform all aerosol tests. The device is primed by first inserting the blister pack into the device, pulling out the device handle and then compressing the chamber by depressing the handle to pressurize the device. The device is actuated by pushing the button that raises the blister pack, punctures it and disperses the powder into the chamber of the device forming an aerosol cloud. All of the filled blister packs were stored in the dry box until use for aerosol testing.

Emitted Dose Aerosol was collected on a glass fiber filter placed in a holder over the mouthpiece of the chamber of the device. To measure the emitted dose percent (ED %), a blister pack was dispersed as an aerosol using the device and the powder sample was collected on a pre-weighed glass fiber filter (GELMAN™, 47 mm diameter) by drawing the aerosol from the chamber at an airflow rate of 30 L/min for 2.5 seconds, controlled by an automatic timer. This sampling pattern simulates the patients' slow deep inspiration. The ED % was calculated by dividing the mass of the powder collected on the filter by the mass of powder in the blister pack. Each result reported was the average and standard deviation of 10 measurements (Table 5).

Particle Size An 8-stage (9.0, 5.8, 4.7, 3.3, 2.1, 1.1, 0.7, and 0.4 μm pore sizes) cascade impactor (ANDERSEN CASCADE IMPACTOR™) was used to measure particle size distribution. Each measurement was obtained by dispersing 5 blister packs of 5 mg fill weight in the device. A vacuum was pulled through the impactor at the calibrated flow rate of 28.5 L/min for 2.5 seconds, controlled by an automatic timer (Table 5). The MMAD is the midpoint or median of the aerodynamic particle size distribution of an aerosolized powder determined by cascade impaction.

The FPF_{%<3.3 μm} was also obtained using the cascade impactor. Fine Particle Fraction_{%<3.3} μm is the total mass under stage 3 of the Andersen impactor when operated at a flow rate of 1 cubic feet per minute (cfm) (28.3 L/min) only. The summed masses from stages 4, 5, 6, 7 and the 8 divided by the total mass collected on all stages is the reported value.

TABLE 5a
Study 1 Aerosol Tests at Initial and Three Weeks Storage

| | Initial | | | Three Weeks |
|---|---|---|---|---|
| | t = 0 | | | Storage |
| | | MMAD | FPF | 40° C./75% RH |
| Lot # | ED ± *RSD (%) | (μm) | (% < 3.3 μm) | ED ± RSD (%) |
| 8 | 21.0 ± 15% | 5.0 | 18 | 22.9 ± 5% |
| 9 | 36.6 ± 9% | 3.4 | 48 | 35.2 ± 10% |
| 10 | 62.2 ± 9% | 3.3 | 50 | 51.0 ± 8% |
| 11 | 13.6 ± 10% | 3.7 | 43 | 16.1 ± 9% |
| 12 | 19.7 ± 15% | 3.4 | 48 | 25.0 ± 14% |
| 13 | 19.4 ± 12% | 3.4 | 49 | 23.0 ± 24% |

*RSD = standard deviation/mean × 100

TABLE 5B
Study 2 Aerosol Tests at Initial and Two Weeks Storage

| | Initial | | | Two weeks | | | | | |
|---|---|---|---|---|---|---|---|---|---|
| | t = 0 | | | 25° C./75% RH | | | 40° C./75% RH | | |
| | ED ± RSD | MMAD | FPF | ED ± RSD | MMAD | FPF | ED ± RSD | MMAD | FPF |
| Lot # | (%) | (μm) | (% <3.3 μm) | (%) | (μm) | (% <3.3 μm) | (%) | (μm) | (% <3.3 μm) |
| 3 | 57.3 ± 5% | 3.4 | 49 | n/a | n/a | n/a | 49.7 ± 5% | n/a | n/a |
| 4 | 62.2 ± 6% | 4.2 | 36 | n/a | n/a | n/a | 53.9 ± 6% | 4.2 | 37 |
| 5 | 77.9 ± 3% | 2.8 | 60 | n/a | n/a | n/a | 68.0 ± 6% | 2.4 | 73 |
| 6 | 89.0 ± 5% | 2.9 | 58 | 90.4 ± 10% | 2.7 | 64 | 80.7 ± 7% | 2.9 | 60 |
| 7 | 50.1 ± 1% | 3.5 | 44 | 52.4 ± 12% | 3.8 | 40 | 46.8 ± 9% | 3.6 | 42 |

Morphology Scanning Electron Microscopy was utilized to obtain initial morphological information on the spray dried powders and to assess changes in morphology after stability. All samples were prepared in a glovebox at relative humidity less than 5%. Samples were mounted on silicon wafers mounted on top of double-sided carbon tape on an aluminum SEM stub. The mounted powders were then sputter-coated in a Denton sputter coater for 60-90 seconds at 75 mTorr and 38 mA with gold:palladium. This produces a coating thickness of approximately 150 Å. Images were taken with a Philips XL30 ESEM operated in high vacuum mode using an Everhart-Thomley detector to capture secondary electrons for the image composition. Accelerating voltage was 3 to 10 kV using a LaB₆ source. Working distance is approximately 5 μm.

All powders except lot 4 (neat formulation in ethanol) did not show any appreciable changes in morphology after either 2 or 3 weeks storage at the temperature and RH conditions described in the stability protocol. The ethanol powders exhibited morphological changes at 40° C. at 75% RH. At the accelerated storage condition the ethanol formulations was more wrinkled and contained some fragmentation when compared to initial.

Residual Solvent The residual solvent content in the powder after spray drying was determined by TGA using a TA INSTRUMENTS™ (New Castle, Del.) TGA. Approximately 3 mg of powder was packed into a hermetically sealed aluminum pan in a glovebox with a relative humidity less than 5%. Prior to analysis the pan was punctured with a pin and loaded onto the equipment. The method used was 110° C./min. run from room temperature to 175° C. (Table 6).

TABLE 6a
Study 1 Solvent Content (wt %)

| | | 3 Weeks Storage | | | | |
|---|---|---|---|---|---|---|
| | Initial | | 25° C./ | 25° C./ | 25° C./ | 40° C./ |
| Lot # | t = 0 | 2-8° C. | 0% RH | 33% RH | 75% RH | 75% RH |
| 8 | 1.8 | 2.6 | 3.7 | 4.4 | 9.5 | 4.2 |
| 9 | 1.7 | 3.1 | 2.8 | 4.6 | 14.1 | 3.6 |
| 10 | 1.6 | 2.6 | 2.1 | 3.7 | 9.5 | 2.6 |
| 11 | 2.0 | 3.3 | 3.1 | 5.7 | 11.8 | 4.1 |
| 12 | 1.8 | 3.3 | 2.5 | 3.9 | 11.8 | 3.9 |
| 13 | 3.6 | n/a | n/a | n/a | n/a | n/a |

TABLE 6b
Study 2 Solvent Content (wt %)

Two Weeks Storage

| | Initial | 25° C./ | 25° C./ | 40° C./ | 40° C./ |
|---|---|---|---|---|---|
| Lot # | t = 0 | 0% RH | 75% RH | 0% RH | 75% RH |
| 3 | 7.1 | n/a | n/a | n/a | n/a |
| 4 | 3.9 | 4.9 | 25.2 | 6.2 | 12.7 |
| 5 | 4.2 | 3.3 | 13.5 | 4.0 | 4.8 |
| 6 | 3.2 | 4.2 | 10.6 | 7.1 | n/a |
| 7 | 3.8 | n/a | 25.6 | 4.7 | 14.2 |

Protein Stability Several techniques were used to analyze samples for aggregation and degradation. Soluble aggregates were measured quantitatively by SE-HPLC. The HPLC was a WATERS™ system, Alliance model 2690. The chromatography system was equipped with a solvent delivery system, a photo diode array detector, a temperature controlled autosampler and data management system. Mobile phase consisted of 50 mM sodium phosphate with 150 mM sodium chloride adjusted to pH 7.0, running isocratically at 1 mL/min. The column was a TOSOHAAS™ TSK G3000SWXL column, 7.8×300 mm, 5 μm pore size with a guard column. Samples were either reconstituted or diluted to a concentration of 1 mg of rF.IX peptide/mL with water. Samples were stored at 5° C. until injection. Chromatograms were extracted and processed at 214 nm. The percentage monomer content of the formulated solutions; before spray drying were compared to the corresponding reconstituted aerosol drug powders.

UV spectrophotometric analyses were used to evaluate turbidity (aggregation/precipitation) in samples. Measurements were performed on a HITACHI™ U-3000, dual beam spectrophotometer. Instrument parameters were set at a scan rate of 300 nm/min; 1.0 nm slit width; and a scan range from 400 nm to 200 nm. Samples were visually inspected for particulate matter. Insoluble aggregates were determined quantitatively by measuring the turbidity of the solution with UV. Linear regression to correct for scatter was performed from absorbance values at 350, 375 and 400 nm. Absorbance at λmax corrected for light scattering was extrapolated from the equation for the regression line. The percent insoluble aggregate is the percentage of absorbance corrected for light scattering, divided by absorbance uncorrected at λₘₐₓ as shown in Eq. 1:

$$\% insoluble aggregates = \frac{{Abs}_{λ max} ( light scatter corrected )}{{Abs}_{λ max} ( light scatter uncorrected )}$$

A value of less than 5% insoluble aggregation was set as the criteria for indication of formulation stability. Samples were either reconstituted or diluted to a concentration of 0.1 mg of rF.IX peptide/mL with water.

All solution samples before (pre-SD) and after spray drying did not have any visible signs of particulate matter or had less than 5% insoluble aggregates. All samples in Study 1 and Study 2 placed at temperature and humidity stability did not exhibit any visible signs of particulates or detectable insoluble aggregates. Less than 5% insoluble aggregates were calculated using Eq. 1 for all batches. Therefore, Table 7 is data collected only by SE-HPLC.

TABLE 7a
Study 1 Monomer Content %

| | | | | 25° C./ | | 25° C./ | | 25° C./ | |
|---|---|---|---|---|---|---|---|---|---|
| Lot # | pre-SD | 2-8° C. | | 0% RH | | 33% RH | | 75% RH | |
| / | / | t = 0 | 3 wk | t = 0 | 3 wk | t = 0 | 3 wk | t = 0 | 3 wk |
| 8 | 99.0 | 98.7 | 98.6 | 98.7 | 98.1 | 98.7 | 97.1 | 98.7 | 96.6 |
| 9 | 99.1 | 96.1 | 95.2 | 96.1 | 94.7 | 96.1 | 87.8 | 96.1 | 93.7 |
| 10 | 99.2 | 98.4 | 97.2 | 98.4 | 94.5 | 98.4 | 96.3 | 98.4 | 93.6 |
| 11 | 99.1 | 96.3 | 95.2 | 96.3 | 98.2 | 96.3 | 97.3 | 96.3 | 96.6 |
| 12 | 97.87 | 97.7 | 96.7 | 97.7 | 95.7 | 97.7 | 95.0 | 97.7 | 93.3 |
| 13 | 83.0 | 80.7 | n/a | 80.7 | n/a | 80.7 | n/a | 80.7 | n/a |

TABLE 7b
Study 2 Monomer Content %

| | | 25° C./ | | 25° C./ | | 40° C./ | | 40° C./ | |
|---|---|---|---|---|---|---|---|---|---|
| | | 0% RH | | 75% RH | | 0% RH | | 75% RH | |
| Lot # | pre-SD | t = 0 | 2 wk | t = 0 | 2 wk | t = 0 | 2 wk | t = 0 | 3 wk |
| 3 | 97.9 | 97.6 | n/a | 97.6 | 94.7 | 97.6 | 96.7 | 97.6 | n/a |
| 4 | 97.6 | 94.8 | 94.6 | 94.8 | 91.4 | 94.8 | 93.3 | 94.8 | 72.4 |
| 5 | 97.6 | 94.7 | 94.1 | 94.7 | 72.0 | 94.7 | 89.5 | 94.7 | 61.4 |
| 6 | 97.7 | 96.9 | 97.1 | 96.9 | 90.4 | 96.9 | 96.3 | 96.9 | 76.3 |
| 7 | 97.9 | 97.6 | 97.7 | 97.6 | 97.7 | 97.6 | 96.7 | 97.6 | 71.4 |

Soluble aggregates and degradation were measured qualitatively by SDS-PAGE. NOVEX™ pre-cast 4-20% tris-glycine gels were run on a NOVEX XCELL II™ electrophoresis mini-cell. Samples were either reconstituted or diluted to a concentration of 0.1 mg of rF.IX peptide/mL with water. Solutions were prepared under reducing and non-reducing conditions to deliver a load of 1 μg of protein to each lane. Reduced samples were treated with 2-mercaptoethanol. Gels were run at 125V, 25 mA per/gel until the gel front reached the bottom (approx 1.5 hrs). Silver staining detection was used for increased sensitivity using a NOVEX SILVER XPRESS™ staining kit. Reducing and non-reducing gels were prepared using samples from both study 1 and 2 representative formulations at stability time points of 2 weeks, 25° C. and 2 weeks, 40° C. The intent of running these gels was to overload the lanes with a 5 μg protein load to detect any faint bands not found in the 1 μg protein load.

There were no changes in the gel profiles between the formulated solutions, before spray drying and the reconstituted aerosol drug powders (data not shown). The monomer band of all samples and controls of rF.IX on the gels were running at a higher molecular weight (approx. 65 kDa) than reported values and appears broad and diffused. This is most likely attributed to the protein being glycoslyated and effecting the migration of rF.IX through the gel. Besides the monomer band, there were other bands that were attributed to rF.IXa and c-terminal peptide. However there were no difference between before spray dry and reconstituted aerosol drug powders.

Summary After selecting a lower atomization pressure on the second screening experiment, the aerosol performance of the rF.IX powder formulations met the project objectives with the trileucine formulation performing the best on all accounts. The emitted doses were 57, 62, 78, 89 and 50% and the aerosol MMAD values were 3.4, 4.2, 2.8, 2.9 and 3.5 μm with 49, 36, 60, 58 and 44% less than 3.31 μm for the neat rF.IX, 5% ethanol to rF.IX in citrate, 60% leucine to rF.IX in citrate, 40% trileucine to rF.IX in citrate and neat rF.IX heated to 37° C., respectively.

In study 2, the ethanol and leucine formulations each had a 3% drop in monomer content comparing the pre-spray dried solution to the reconstituted aerosol drug powder at initial. There were no changes in the other formulations in study 2 at initial compared to pre-spray dried. Based on two weeks stability study, humidity had the greatest affect on chemical stability as measured by SE-HPLC. No insoluble aggregation was observed by UV for all the batches. No extra soluble aggregates or degradation bands were observed using SDS-PAGE. Clotting activity of select formulations were not compromised due to spray drying. Activity after spray drying was an average of 80-90% of the activity prior to spray drying, as measured by F.IX assay, with the best formulations performing at 95% or better.

The ethanol (lot 4) spray dried powder was the only formulation that demonstrated morphological changes as observed by SEM. At 2 weeks stability at 40° C./75% RH, the ethanol formulation was more wrinkled and contained fracture fragments. No significant morphology changes were noted on any of the other powders when exposed to identical storage conditions. This data suggests that dry F.IX suitable for pulmonary delivery should not be spray dried with alcohol.

### EXAMPLE 3

### In Vivo Bioavailability Study

The first two studies showed 1) that efficacious levels of liquid rF.IX could be systemically delivered via the intratracheal surfaces, and 2) that dry powder rF.IX could be successfully aerosolized, while maintaining enzymatic activity and stability. The next experiment employed Formulation 6 (tri-leucine excipient) in an in vivo dog model to test for bioavailability of the rF.IX.

The objectives of this study were to determine the pharmacodynamic and pharmacokinetic parameters of human Factor IX after oral inhalation in hemophilia B dogs that had been previously tolerized to human Factor IX. The data from this study is compared against data from a subsequent study administering human Factor IX by intravenous injection. Parameters that were measured included 1) whole blood clotting time (WBCT), 2) F.IX antigen (F.IX:Ag), 3) activated partial thromboplastin time (APTT), 4) F.IX activity, 5) F.IX antibodies by ELISA, and 6) the Bethesda inhibitor Assay.

Dogs: Five hemophilia dogs from the Chapel Hill colony (see Example 1) were used in the study. Of the five dogs used, four were human F.IX tolerized hemophiliac dogs and were on prophylaxis (82 IU/Kg SC on Monday and Thursday). Two dogs did not receive their last dose (Thursday) in lieu of day 1 dosing by inhalation. One dog was naive to rF.IX.

In order to evaluate aerosol delivery in the dog model, a modified device as described in U.S. Pat. No. 6,257,233 was used. Briefly, air was provided by compressed air (˜5 psi), through a regulator, HEPA filter and a series of valves. A personal computer (PC) regulated the flow of air through the system. Air was delivered to the device as modified, and to the dogs lungs through an ET tube, which was held in place via a cuff. A relief valve prevented too much air from being delivered, and a U-manometer monitored the pressure of the delivered air.

A computer was used to control known volume of compressed air (˜800 ml) and the flow rate. The compressed air was used to deliver aerosol to the dog through the endotracheal tube. The volume generated by this system was based on the lung mechanics of an anesthetized 10 kg dog. The total maximum lung volume of an anesthetized dog is about 1400 ml, and the average delivery bolus was 800 ml.

The catheters were placed in the dog on the day of the study using the following procedure. For general anesthesia the animal was sedated using Thiopental Na to effect. The animal was intubated and isoflurane used to maintain anesthesia (2-4% inhaled with supplemental oxygen). The animal was evaluated for heart rate, respiration rate, blood pressure, and persistence or absence of palpebral, comeal, and withdrawal reflexes. For procedures requiring local anesthesia and sedation, dogs received Meditomidine, Valium, Butorphanol Tartrate, or Propofol or a similar analgesic/sedative.

The dogs were hyperventilated for 1-4 min using 2% isoflurane & oxygen. This results in apnea of approximately 3 minute duration. During the period of apnea the dog was connected to the aerosol apparatus and 800 ml boluses of air given through the system. The aerosol delivery of the system was pre-characterized using a laser, an in-line filter, and a balloon to simulate a dog's lung. It was concluded that most of the aerosol bolus delivered at about 600 ml.

For comparison, dogs received an intravenous injection of recombinant human F.IX at comparable doses with the same sampling and analysis protocol. This protocol was initiated at least 28 days after the inhalation study completion. All of these dogs have had similar IV boluses in the past as part of their characterization.

TABLE 3
Group Assignments

| | | | | Effective | No. of | | Total |
|---|---|---|---|---|---|---|---|
| Group | Test | Route of | No. of animals | dose^{a} rF.IX | Blisters per | No. of | no. of |
| no. | Article | Administration | and Gender | Ul/kg | actuation | actuation | blisters/dog |
| 1 | Factor IX | OI | 2M & 2F | 50 | 3 | 2 | 6 |

^{a}Effective dose may vary due to delivery efficiency.

Dosage: Recombinant Human Factor IX was supplied as a blister pack containing 7.5 mg by weight of a powder, of which 3.95 mgs is glycoprotein, 0.55 mgs NaCitrate, and 3.0 mgs of an excipient (tri-leucine), pH 6.4. Each 7.5 mg blister will deliver approximately 5 mg of powder. The specific activity is approximately 300 units/mg protein. For every 1.0 mg of glycoprotein, 85.5% is protein and the remainder is the sugar moiety.

Sample Collection: Blood was collected from the jugular or cephalic vein at the following time points listed below. For determining the plasma concentration of Factor IX antigen and APTT, blood samples (3.0 ml) were collected into 3.8% citrate containing tubes at the following time points: immediately prior to dosing, 0.08, 0.25, 0.5, 1, 2, 4, 6, 8, 12, 24, 28, 32, 48, 72, and 96 hours post-dose. Additional blood samples were collected at predose, and immediately prior to the Monday subcutaneous dose, every week for 4 consecutive weeks for determining the formation and concentration of antihuman Factor IX antibodies.

The plasma was separated by centrifuging at 4500 rpm for 15 minutes at 4° C. Serum was separated by centrifuging at 3000 rpm for 15 minutes at room temperature. The plasma was divided into at least three aliquots into 12×75 mm polypropylene cryovials. All plasma/serum containing tubes were frozen at approximately −80° C. until needed.

Data Analysis: Pharmacokinetic analysis of the plasma concentrations of Factor IX was performed to determine parameters such as the maximum plasma concentrations (Cmax), time to maximum plasma concentration (Tmax), areas under the plasma concentration vs. time curve (AUC), and apparent elimination half-life (t½). Analysis was preformed using WINNONLIN PROFESSIONAL 2.0™ (SCIENTIFIC CONSULTING™, Apex, N.C.) validated computer program or equivalent. In addition, the plasma concentration of APTT and antibody concentration was plotted against time.

F.IX Bioassay: Factor IX (F.IX) coagulant activities were determined by a modified one-stage partial thromboplastin time assay using canine F.IX deficient substrate plasma. Normal human reference plasma consists of pools from 5-10 normal humans. The test sample was diluted several fold and compared to the same dilutions for a normal curve. The results are reported as a percent of normal.

APTT: APTT was determined with the ST4™ coagulation instrument (DIAGNOSTICA STAGO™, Asnieres, France) or the MULTIPLE DISCRETE ANALYZER (MDA) 180™ (ORGANON TEKNIKA™) that has the capacity to process rapidly a large number of samples. Whether the APTT's are determined on the ST4™ coagulation instrument or the MDA 180™, the controls and reagents are of the same type. For the APTT test, mixtures consisted of equal portions of partial thromboplastin (AUTOMATED APTT™, ORGANON TEKNIKA™), 0.025 M CaCl₂, and citrated test plasma.

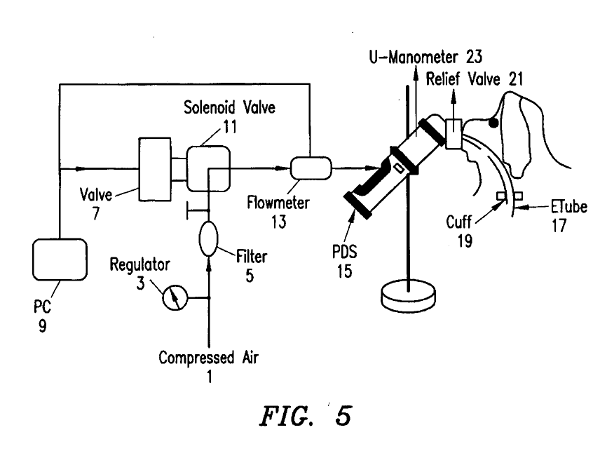

The results are shown in FIG. 5. The APTT shortened from 90 seconds to 70-75 seconds for about 100 hours after inhalation dosing. This is typical for a low dose prophylactic response.

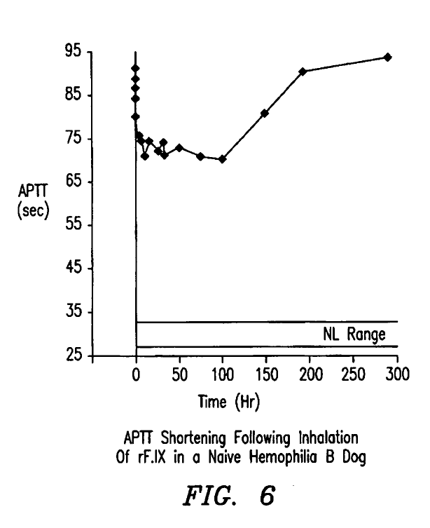

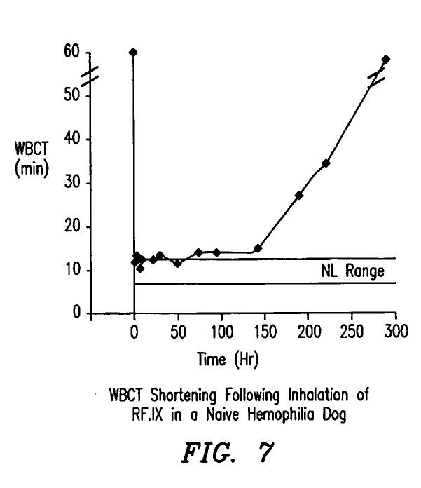

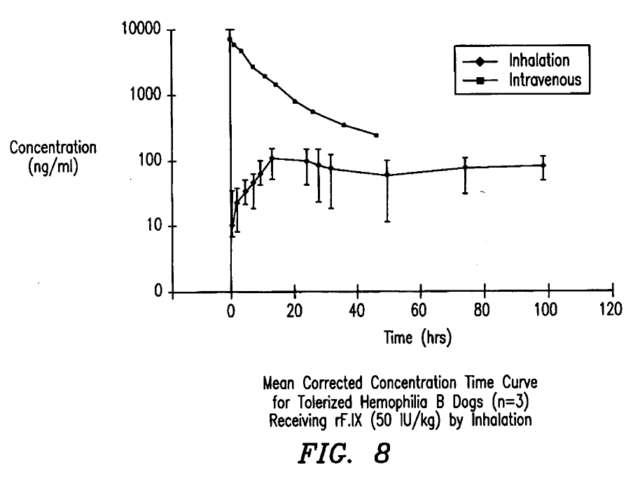

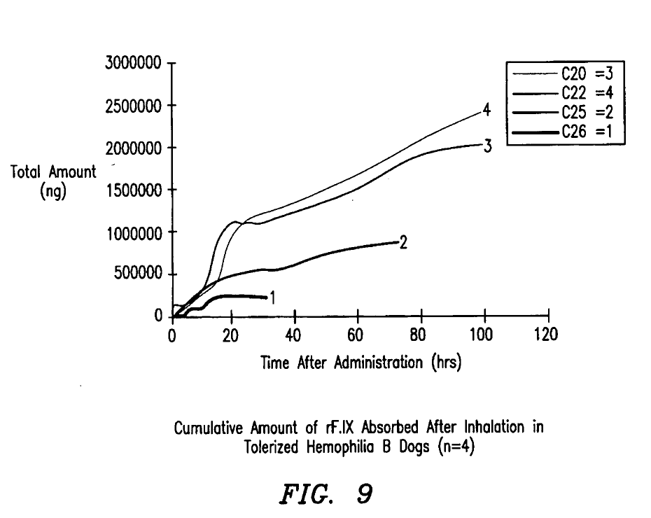

WBCT: The WBCT was performed as previously described (7, 13-15). The WBCT is typically greater than 50 min in untreated hemophilia B dogs from the Chapel Hill colony (14, 15). The reference range for WBCT in normal, healthy dogs in this colony is 8 to 12 min. The results are shown in FIG. 6. The WBCT reduced from 50+minutes to around 10 minutes.

Bethesda Inhibitor Assay: The Bethesda Inhibitor assay for Factor IX was performed with the Nijmegan modifications to the procedure originally reported by Kasper et al. (34, 35). Briefly, a patient's plasma with a residual Factor IX activity of 50% of the normal control is defined as one Bethesda unit (BU) of inhibitor per mL. Appropriate screening dilutions was made to detect both low titer (2 BU) and high titer (>5 BU) inhibitors. No inhibitors were found (data not shown).

Factor IX antigen: Antigen concentration was determined using a double monoclonal antibody sandwich enzyme-linked immunosorbant assay (ELISA) by Genetics Institute.

### EXAMPLE 4

### Factor VIII

Factor VIII is also important in the treatment of hemophilia A and Factor XI for the treatment of Factor XI deficiency. Experiments are planned to confirm that F.VIII can be also delivered by aerosol inhalation therapy, as is described above for Factor IX. FVIII will be aerosolized as described in Example 2, using the same formulations and the method of study 2.

All references cited herein are expressly incorporated by reference for all purposes:
1. Liles D, et al., Extravascular administration of Factor IX: Potential for replacement therapy of canine and human hemophilia B. Thromb Haemost 1997; 77: 944-8.
2. Patton J S, Platz R M. Pulmonary delivery of peptides and proteins for systemic action. Adv Drug Del Rev 1992; 8: 179-96.
3. Hubbard R C, et al., Fate of aerosolized recombinant DNA-produced alpha1-antitrypsin: Use of epithelial surface of the lower respiratory tract to administer proteins of therapeutic importance. Proc Natl Acad Sci 1989; 86: 680-4.
4. Brinkhous K M, et al., Recombinant human Factor IX: Replacement therapy, prophylaxis, pharmacokinetics, and immunogenicity in canine hemophilia B. Blood 1996; 88: 603-10.
5. Russel K E, et al., Intratracheal administration of recombinant human Factor IX (BeneF.IX™) achieves therapeutic levels. Blood 1998; 92: 356a.
6. Evans J P, et al., Canine Hemophilia B resulting from a point mutation with unusual consequences. Proc Natl Acad Sci USA 1989; 86: 10095-9.
7. Kay M A, et al., In vivo gene therapy of hemophilia B: Sustained partial correction in Factor IX-deficient dogs. Science 1993; 262: 117-9.
8. Herzog R, et al., Absence of circulating Factor IX antigen in hemophilia B dogs of the UNC Chapel Hill Colony. Thromb Haemost 2000; 84: 352-4.
9. Harrison S, et al., Development of a serum-free process for recombinant Factor IX expression in Chinese hamster ovary cells. Thromb Haemost 1995; 73: 1222.
10. Foster W B, et al., Development of a process for purification of recombinant human Factor IX. Blood 1995; 86: 870a.
11. Rodriques H, et al., Analytical characterization of recombinant human Factor IX: Pharamacokinetic studies in the rat and the dog. Thromb Haemost 1995; 73: 1206.
12. Keith J C, et al., Evaluation of recombinant human Factor IX: Pharmacokinetic studies in the rat and the dog. Thromb Haemost 1994; 73: 101-5.
13. Lee R I, White P D. A clinical study of the coagulation time of blood. Am J Med Sci 1913; 145: 495-503.
14. Brinkhous K M, et al., Evaluation of sensitivity of whole blood clotting time (WBCT), partial thromboplastin time (PTT), and F.IX one-stage bioassay tests with low plasma F.IX levels observed with transfusion or gene therapy in canine hemophilia B. Blood 1993; 82 (Suppl 1): 592a.
15. Mustafa R L, et al., A role for whole blood clotting time as a sensitive screening test for F.VIII and F.IX replacement. Blood 1998; 92: 96b.
16. Pedersen P V. Novel Deconvolution Method for Linear Pharmacokinetic Systems with Polyexponential Impulse Response. J Pharm Sci 1980; 69:312-8.
17. Gerrard A J, et al., Subcutaneous injection of Factor IX for the treatment of haemophilia B. British Journal of Haematology 1992; 81: 610-3.
18. McCarthy K P, et al., Subcutaneous bioavailability of recombinant Factor IX (BeneF.IX™) in healthy beagle dogs and cynomolugus monkeys. Blood 1996; 88: 68b.
19. Cheung W F, et al., Identification of the endothelial cell binding site for Factor IX. Proc Natl Acad Sci USA 1996; 93: 11068-73.
20. Wolberg A S, et al., Human Factor IX binds to specific sites on the collagenous domain of collagen IV. J Biol Chem 1997; 272: 16717-20.
21. Brinkhous K M, et al., Subcutaneous recombinant Factor IX (BeneF.IX™) administration produces therapeutic levels of F.IX activity in hemophilia B dogs. Blood 1996; 88: 422a.
22. Yu J, Chien Y W. Pulmonary drug delivery: Physiologic and mechanistic aspects. Crit Rev Therap Drug Carr Sys 1997; 14: 395-453.
23. Brain J D, et al., Pulmonary distribution of particles given by intratracheal instillation or by aerosol inhalation. Environ Res 1976; 11: 13-33.
24. Colthorpe P, et al., The pharmacokinetics of pulmonary-delivered insulin: A comparison of intratracheal and aerosol administration to the rabbit. Pharm Res 1992; 9: 764-8.
25. Gillespie M N, et al., Pulmonary metabolism of exogenous enkephalins in isolated perfused rat lungs. J Pharmacol Exp Ther 1985; 232: 675-8.
26. Jendle J, et al., Delivery and retention of an insulin aerosol produced by a new jet nebulizer. J Aersol Med 1995; 8: 243-54.
27. Jendle J H, et al., An exploration of intrapulmonary insulin administration in anaesthetized and mechanically ventilated pigs. Scand J Clin Lab Invest 1996; 56: 251-8.
28. Laube B L, et al., Preliminary study of the efficacy of insulin aerosol delivered by oral inhalation in diabetic patients. JAMA 1993; 269: 2106-9.
29. Gupta S, et al., Pulmonary delivery of human protein C and Factor IX, in (ed) NaL (ed): Oxygen Transport to Tissue XVIII. New York: Plenum Press 1997; p 429-35.
30. Wigley F M, et al., Insulin across respiratory mucosae by aerosol delivery. Diabetes 1971; 20: 552-6.
31. Lusher J M. Thrombogenicity associated with Factor IX complex concentrates. Semin Hematol. 1991;28(3 supp. 6):3-5.
32. Shapiro A D, Ragni M V, Lusher J M, et al. Safety and efficacy of monoclonal antibody purified IX concentrates in previously untreated patients with hemophilia B. Thromb Haemost. 1996;75(1):30-35.
33. Roberts H R, Eberst M E. Current management of hemophilia B. Hemat Oncol Clin North Am. 1993;7(6):1269-1280.
34. Kasper C K, et al. A more uniform measurement of factor VIII inhibitors. Thromb Diathes Haemorrh 34:869-872, 1975.
35. Giles A R, A detailed comparison of the performance of the Standard versus Nijmegan modifications of the Bethesda Assay in detecting factor VIII. Thromb. Haemost 79:872-5, 1998.
36. U.S. Pat. No. 6,518,239, U.S. Pat. No. 6,372,258

1) A method of treating hemophilia, said method comprising
a) aerosolizing a Factor IX (F.IX), wherein the aerosolized F.IX:
i) has a mass median aerodynamic diameter (MMAD) of between 2 and 4 μm, has a fine particle fraction percent less than 3.3 μm (FPF_{%<3.3 μm}) of at least 50%,
ii) is at least 90% monomeric,
iii) wherein the after-aerosolization activity/pre-aerosolization activity is at least 80%; and
iv) is a dry powder having less than 10% water (wt/wt);
b) inhaling the aerosolized F.IX and allowing the aerosolized F.IX to deposit in the lung;
c) followed by exhalation.

2) The method of claim 1, wherein the MMAD is 2.8 to 3.6 μm, the FPF_{%<3.3 μm} is at least 60%, the monomer content is at least 95% and the after-aerosolization activity/pre-aerosolization activity is at least 90%.

3) The method of claim 1, wherein the MMAD is about 3-3.5 μm, the FPF_{%<3.3 μm} is at least 64%, the monomer content is at least 97%, and the after-aerosolization activity/pre-aerosolization activity is at least 95%.

4) The method of claim 1, wherein the F.IX is aerosolized without alcohol.

5) The method of claim 1, wherein the F.IX is recombinant.

6) The method of any of claims 1 through 5, wherein the F.IX comprises a tri-leucine excipient.

7) The method of claim 6, wherein the tri-leucine/F.IX ratio is 0.5-1.5 wt/wt.

8) A method of treating hemophilia, said method comprising the inhalation of aerosolized, dry Factor IX (F.IX), wherein the aerosolized dry F.IX:
a) comprises a surface active di- or tri-peptide, b) has a MMAD of between 2.8-3.5 μm, c) an FPF_{%<3.3 μm} of greater than 60%, d) a monomer content of at least 95%, e) the after-aerosolization activity/pre-aerosolization activity is at least 80%, and f) less than 10% water.

9) The method of claim 8, wherein the MMAD is about 3-3.5 μm, the FPF_{%<3.3 μm} is at least 64%, the after-aerosolization activity/pre-aerosolization activity is at least 90%; the monomer content is at least 97% and the water content is less than 5%.

10) The method of claim 8, wherein the F.IX does not contain alcohol.

11) The method of claim 8, wherein the F.IX is recombinant.

12) The method of any of claims 8 through 11, wherein the F.IX comprises a tri-leucine excipient.

13) The method of claim 6, wherein the tri-leucine/F.IX ratio is 0.5-1.5 wt/wt.

14) A method of preventing hemophilic bleeding in advance of a hemophilic assault, said method comprising
a) aerosolizing a Factor IX (F.IX), wherein the aerosolized F.IX:
i) has a mass median aerodynamic diameter (MMAD) of between 2 and 4 μm,
ii) has a fine particle fraction percent less than 3.3 μm (FPF_{%<3.3 μm}) of at least 50%,
iii) is at least 90% monomeric,
iv) wherein the after-aerosolization activity/pre-aerosolization activity is at least 80%; and
v) is a dry powder having less than 10% water (wt/wt);
b) inhaling the aerosolized F.IX at least once per week and allowing the aerosolized F.IX to deposit in the lung;
c) followed by exhalation.

15) The method of claim 14, wherein the inhalation is bi-weekly.

16) The method of claim 14, wherein the inhalation is every 2 to 3 days.

17) A composition comprising aerosolizable dry F.IX having, when aerosolized an MMAD between 2 and 4 μm, an FPF_{%<3.3 μm} of at least 50%, an emitted dose (ED) of at least 50%, a monomer content of at least 95%, wherein the after-aerosolization activity/pre-aerosolization activity is at least 80%, less than 10% water, and a surface active di- or tri-peptide excipient, but does not have ethanol.

18) The composition of claim 17, wherein the MMAD is between 2.8 and 3.6 μm, the ED is at least 60%, the after-aerosolization activity/pre-aerosolization activity is at least 95%, the FPF_{%<3.3 μm} is at least 65% and less than 5% water.

19) The composition of claim 17, wherein the MMAD is between 3 and 3.5 μm, the FPF_{%<3.3 μm} is at least 64%, the ED is at least 80%, wherein the after-aerosolization activity/pre-aerosolization activity is at least 95%, the monomer content is at least 97% and the water content is less than 5%.

20) A blister pack containing F.IX, wherein the blister pack is waterproof and contains F.IX that is at least 90% monomeric and has less than 10% (wt/wt) water and a surface active di- or tri-peptide excipient, but does not have ethanol.

21) The blister pack of claim 20, wherein the F.IX is at least 95% monomeric and has less than 5% (wt/wt) water and the excipient is a dileucyl or a tri-leucine.

22) The blister pack of claim 20, wherein the F.IX is at least 97% monomeric and has less than 5% (wt/wt) water and the excipient is tri-leucine.

23) The blister pack of any of claims 20 to 22, wherein the F.IX is recombinant F.IX.

24) A dry powdered F.IX comprising a biologically active recombinant Factor IX that is at least 90% monomeric and has less than 10% water, and a surface active di- or tri-peptide excipient, but does not have ethanol.

25) The dry powdered F.IX of claim 24, wherein the excipient is tri-leucine.

26) The dry powdered F.IX of claim 25, wherein there ratio of F.IX to excipient is 0.2-5.0/1.

27) A composition comprising dry, dispersible powder and a solid content of about 50 wt % glycosylated F.IX, about 40 wt % trileucine and about 10 wt % buffer.

28) A composition comprising dry, dispersible powder and a solid content of 40-60 wt % glycosylated F.IX, 40-60 wt % trileucine and 0-10 wt % buffer.
